(19) United States
(12) Patent Application Publication
(10) Pub. No.: US 2006/0047183 A1
(43) Pub. Date: Mar. 2, 2006

(54) Inflatable guide device

(75) Inventor: Chul Hi Park, Seoul (KR)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Chul Hi Park, Seoul (KR)

(21) Appl. No.: 11/213,285

(22) Filed: Aug. 26, 2005

Related U.S. Application Data
(60) Provisional application No. 60/606,640, filed on Sep. 1, 2004.

(51) Int. Cl. A61B 1/00

(52) U.S. Cl. 600/114

(57) ABSTRACT

A guide device for an endoscope comprises a tubular member that is configured to be inflated and collapsed; a plurality of straight portions; and a plurality of looped portions. The plurality of straight portions and the plurality of loop portions are arranged in an alternating manner and together comprise the tubular member. The guide device is configured to receive an endoscope and guide the endoscope along a path within an organ.

### CROSS REFERENCE TO RELATED APPLICATONS

The present application claims priority to U.S. Provisional Application No. 60/606,640, filed on Sep. 1, 2004, which is incorporated by reference.

### BACKGROUND OF THE INVENTION

This invention relates to an assist device in the insertion into a tortuous passage and maneuver therein of an endoscope and method of use. More particularly, it relates to a device and method to facilitate insertion and maneuver of a flexible endoscope into a tortuous bodily passage, such as the colon, for the examination and treatment.

An endoscope is a medical instrument for visualizing the interior of a patient's body. Endoscopes can be used for a variety of different diagnostic and interventional procedures, including colonoscopy, sigmoidoscopy, bronchoscopy, thoracoscopy, laparoscopy and video endoscopy.

Colonoscopy is a medical procedure in which a flexible endoscope, or colonoscope, is inserted into a patient's colon for diagnostic examination and/or surgical treatment of the colon. A standard colonoscope is typically 135-185 cm in length and 12-19 mm in diameter, and includes a fiber-optic imaging bundle or a miniature camera located at the instrument's tip, illumination fibers, one or two instrument channels that may also be used for insufflation or irrigation, air and water channels, and vacuum channels. A colonoscope is sufficiently stiff so that it does not buckle when it is pushed from proximal end during insertion yet flexible enough to be maneuvered through the tortuous lumen of the colon. The final six inches of the distal end of a colonoscope is usually controllable by operating controls at the end of the colonoscope outside the patient.

The most commonly used procedure for examining the colon is first to insert a colonoscope as far as is desired while inspecting as the colonoscope advances. A detailed examination of the colon is made as the colonoscope is withdrawn. To examine the entire colon, the colonoscope is inserted through the anus into the rectum, and then advanced through the sigmoid colon into the descending colon. The colonoscope then passes through the left colic flexure (the splenic flexure) into the transverse colon, and then through the right colic flexure (the hepatic flexure). The colonoscope next passes through the ascending colon and finally reaches the caecum.

To insert a colonoscope the instrument is grasped at a point outside the body near the anus. The operator pushes the colonoscope inwards and also manipulates the colonoscope so that the inward tip is aimed in the required direction. The colon is difficult to intubate with a flexible colonoscope due to the flexible nature of the colonoscope shaft and the floppy nature of the colon. In some people the sigmoid colon can be very long and is unfixed, except by its mesentery, and so can be extremely difficult to intubate due to its predisposition to form loops when a colonoscope is pushed through it. Some anatomical landmarks, such as the rectosigmoidal junction, the splenic flexure and the hepatic flexure, are difficult to pass through simply because of their tortuous nature. Problems traversing these areas are exacerbated by looping of the colonoscope in the sigmoid colon and subsequent stretching of the sigmoid colon causing discomfort for the patient undergoing the procedure.

The insertion of the endoscope can be uncomfortable for the patient. However, it is generally undesirable to anaesthetize the patient because the patient will be unable to provide feedback to the endoscopist regarding the presence or intensity of pain, which will provide the endoscopist with a helpful indication that the endoscope has been misdirected. There is a risk of perforation of the colon by the advancing colonoscope during insertion even when performed by skilled and experienced endoscopists.

Quite often in a colonoscopy procedure for colorectal cancer screening, the insertion of the colonoscope all the way to caecum takes a lot longer than the withdrawal during which actual inspection of the colon occurs. It is not uncommon screening, diagnostic or therapeutic colonoscopy procedures are abandoned even before the tip of the endoscope reaches the desired depth in the colon because the patient complains intolerable level of pain and further advancement of the endoscope is simply impractical. This leaves diagnosis or treatment of the colon incomplete because a portion of the colon beyond the tip of the endoscope will not have been examined or treated.

From a purely mechanical standpoint, one of the difficulties in intubating the colon with an endoscope originates from the fact that every colon is uniquely shaped. No single standardized approach to intubating a colon could be formulated. Complicating the problem is the fact that the colon is too distensible and the colon wall is too frail to mechanically support the winding movement of an endoscope advancing through the colon's tortuousness. If the colon and the colon wall were much more rigid and tougher, respectively, than they are a force stronger than normally allowable could be applied to advance an endoscope without causing undue discomfort to patient or injury to the colon wall. As a result colonoscopy procedures would become shorter and easier and the success rate would improve.

One solution to the difficulties of colonoscopy would be to provide a means for guiding and supporting the movement of an endoscope inside the colon with suitable mechanical characteristics, which could be readily deployed in and removed from the colon. Preferably, this means would also have to be easy to operate so as not to add another layer of complexity to already complicated procedure. Because only access to the colon is the anus, which would be constricted by the shaft of the endoscope, additionally introducing a conventional guide device into the colon would be nontrivial.

A problem with similar set of underlying constraints, but of much greater scale, has been successfully resolved by employing inflatable structures in the space applications (Holland, D. B., Virgin, L. N., Slade, K. N., and Tinker, M. L., “Geometric Scaling Properties of Inflatable Structures for use in Space Solar Power Generation.”, AIAA Paper 2002-1264, 3rd Gossamer Spacecraft Forum, Denver, Colo., April 2002), which is incorporated by reference. Several large scale structures that were otherwise too heavy or too large for typical payload launch capabilities had been constructed out of collapsible tubes were launched into space in collapsed and folded state of manageable size and weight. The tube structures were then inflated with compressed gas whereupon they took predefined configurations with structural rigidity suitable for intended applications.

Several devices have been described in the prior art to assist in the practice of lower gastrointestinal endoscopy.

U.S. Pat. App. No. 2002/0045906 discloses inflatable sleeve device consisting of an everting sleeve surrounding an endoscope, which is incorporated by reference. The insertion of the endoscope into a passage is assisted by the fluid pressure in the sleeve, which expands the sleeve along the passage drawing the endoscope along. This device is likely to present difficulties negotiating sharp bends found in the tortuous passage. It also has a tendency to develop a buckle especially in and around the sharp bends and the long linear sections of the passage. A buckle in a pressurized sleeve can severely constrict the movement of the endoscope.

U.S. Pat. App. No. 2003/0168068 discloses an inflatable sleeve device consisting of an everting liner and additional means to control the rigidity of the device independent of everting liner, which is incorporated by reference. This device also has a tendency to develop a buckle around sharp bends and in the long straight sections of the passage hindering further movement of the endoscope. Similar problems exist with the device disclosed in U.S. Pat. No. 5,236,423. This device consists of an everting sleeve without additional rigidity control means. The device disclosed in U.S. Pat. App. No. 2002/0016607 is also an everting sleeve device with means to help the device negotiate various curves and bends of the tortuous passage. However, practical implementation of such means is made difficult by the random nature of the tortuousness of the passage. This device also suffers from limitations commonly found in the sleeve devices as described above. U.S. Pat. No. 5,236,423 and App. No. 2002/0016607 are incorporated by reference.

U.S. Pat. App. Nos. 2002/0161281 and 2003/0233066, which are incorporated by reference, describe a guide tube device with controllable rigidity. These devices and an endoscope contained therein are alternately advanced during insertion process. During the movement of the endoscope the guide tube device is held in stiffened state in a shape conforming to the geometry of the passage. These devices are designed to be introduced into the colon over the endoscope through the constricted anus and can cause discomfort for the patient. Usage of these devices allows the endoscope to be advanced only incrementally. These devices employ a complicated locking mechanism for stiffening, which is prone to malfunction and require sterilization after each use.

U.S. Pat. App. No. 2003/0171650, which is incorporated by reference, describes a guide device to be positioned adjacently to the endoscope during insertion in to a tortuous passage. The use and operation of this device is very similar to the guide tube device described above and carries similar disadvantages.

In general, such known devices are either impractical to use, fail to reduce the discomfort to the patient and the time and effort needed for the procedure, or fail to cater for complex nature and shape of the tortuousness of the lower gastrointestinal tract.

### BRIEF SUMMARY OF THE INVENTION

An inflatable guide device and method of use for assisting insertion into a tortuous passage, for example, the colon, and maneuver therein of an endoscope is described herein. The present guide device generally comprises a continuous, collapsible tubular member with looped sections formed at predetermined positions along the length thereof. The present guide device is passively deployed in the colon by advancing action of the endoscope.

In one embodiment, the tubular member comprising the guide device is made of a continuous tube configured to take a predetermined shape when inflated above an ambient pressure. Preferably, in deflated state, the tubular member is soft enough to be pleated into a form with a thin profile while in inflated state it becomes piece-wise rigid. The distal end of the tubular member is sealed in airtight manner and the proximal end is attached to the distal end of the internal supply tube of a supply tube assembly. There is provided a conduit in the supply tube assembly through which a fluid may pass to and from between a pressure controller unit external to the colon and the tubular member to control the internal pressure thereof. Preferably, the fluid is a gas (e.g., air, nitrogen, or carbon dioxide). Alternatively, it may be a low viscosity liquid (e.g., water or saline solution). An endoscope is guided by the openings of the looped sections of the inflated tubular member separated from the colon wall.

Looped sections of the tubular member comprising the guide device of a given embodiment are generally more flexible lengthwise than straight sections. The positions of looped sections along the length of the tubular member may be adjusted to provide different degrees of flexibility at different sections of the tubular member. In an exemplary configuration, several looped sections may be successively joined to produce a spiral configuration, which could fit well around the sharp bends often found in the colon due to its high degree of flexibility. In another exemplary configuration, looped sections may be disposed to alternate with straight sections of predetermined length to make a section of the tubular member more suitable for firmly supporting linear parts of the colon. The straight section may be of any suitable length (e.g., between 20 mm and 60 mm or between 30 mm and 50 mm) when fully inflated. In one implementation the length of straight section is 40 mm. Other arrangements may provide a portion of the tubular member with flexibility somewhere between those of preceding two arrangements.

Looped sections may be of various shapes and sizes. Preferably, the overall dimension of a looped section is predetermined to minimize the amount material needed for construction. Preferably, the size of the opening of a loop is just large enough (e.g., 10 mm and 20 mm or between 13 mm and 17 mm) to allow the endoscope in use to pass freely. In one implementation, the size of the opening is 15 mm. Preferably, the external dimension of a looped section is large enough to provide necessary structural strength to the looped section (e.g., between 15 mm and 40 mm when fully inflated or between 20 mm and 35 mm when fully inflated) In one implementation, the looped section is 25 mm long when fully inflated. Preferably, looped sections with external dimensions large enough to allow the outer side thereof to frictionally engage the colon wall are strategically disposed at predetermined positions to reduce sliding of the colon with respect to the tubular member. The ratio of length to transverse dimension of a section of the tubular member may also be adjusted to effect different degree of flexibility.

The cross-sectional profile of the tubular member may be a circle, a square, a rectangle, an ellipse, or of any other irregular shape. The cross sectional profile has a measurable influence on the flexibility of the thin walled tubular member, which may be advantageously applied to fine-tune the flexibility of different sections of the tubular member. Preferably, the tubular member may have several different cross-sectional profiles along the length thereof. Preferably, the overall length of the guide device is predetermined not to exceed the maximum depth of insertion of the endoscope in a procedure. The dimension of the cross-sectional profile is between 5 mm and 20 mm in the present embodiment, but may be of different dimension according to application. In one implementation, the dimension of the cross-sectional profile is between 8 mm and 15 mm. In another implementation, the dimension of the cross-sectional profile is approximately 10 mm.

In use, the tubular member comprising the present guide device, preferably, is introduced into the colon in a collapsed state stowed in low profile housing. The housing may be detachably mounted, tightly encircling the shaft of the endoscope near the distal end to facilitate the passage of the guide device and endoscope assembly through the constricted anus. Preferably, the tubular member is sequentially released from the housing as the endoscope advances through the colon and immediately inflated by the fluid supplied through the conduit in the supply tube assembly to form a stiffened configuration, which conforms to the shape of the colon. The inflated portion of the tubular member preferably remains stationary with respect to the colon wall while allowing the endoscope to move freely through the openings of the looped sections without exerting undue force on the colon wall. At the end of use the tubular member may be deflated into a collapsed state and withdrawn from the colon.

The housing for the tubular member may be made of a pair of thin polymer film materials. The collapsed portion of the tubular member is stowed in the housing sandwiched by the two polymer films. Preferably, the overall thickness of the housing including the stowed portion of the tubular member is as thin as practically possible to minimize patient's discomfort during its passage through the constricted anus, piggybacking on the endoscope. Preferably, the proximal end of the tubular member comprising the present guide device is attached to and held in position by the internal supply tube. The advancing motion of the endoscope causes the tubular member stowed in the housing to be sequentially released from the proximal end thereof.

When the depth of insertion of the endoscope exceeds the overall length of the tubular member, the distal end of the tubular member leaves the housing completely. On withdrawal of the endoscope, the string fixed to the distal end of the tubular member may be pulled from outside the colon along with the withdrawing endoscope, sequentially deflating the tubular member from the distal end. Alternatively, the distal end of the tubular member may be made to remain tethered to the distal end of the endoscope after leaving the housing so that it could be sequentially deflated in step with the withdrawing endoscope. Preferably, a relief valve attached to the supply tube assembly set at predetermined point keeps the portion of the tubular member, not crumpled by withdrawing action, inflated so that it could continue to guide the endoscope throughout the withdrawal process.

When the endoscope reaches the rectum and the tubular member is fully deflated, the endoscope may be first withdrawn from the colon to allow easy removal of the crumpled tubular member through the anus. After removing the tubular member, the endoscope may be inserted back into the colon to examine the region of the rectum previously blocked from view by the crumpled tubular member. Alternatively, the rectum may be examined prior to commencing the advancement of the endoscope soon after the endoscope is introduced into the rectum. The present guide device may also be left in its fully inflated configuration for repeated insertion and removal of the endoscope during procedures requiring multiple accesses to a site of interest inside the colon.

There are numerous advantages of using the present guide device as an aid for the insertion into a tortuous passage and maneuver therein of an endoscope. For example, the present guide device acts as a stationary and rigid track with a lower friction coefficient than the colon wall on which the shaft of the endoscope travels, thereby reducing force needed to advance or withdraw the endoscope. The colon wall is largely shielded from the moving shaft of the endoscope by the inflated tubular member of the present guide device, which spreads the force exerted by the endoscope over a wide area of the colon wall, substantially reducing possibility of injuries to the mucosal tissue thereof. The structural rigidity inherent to the present guide device effectively resists being stretched or compressed by the force exerted by the flexible shaft of the endoscope, which helps the colon remain close to its natural shape during the insertion and withdrawal of the endoscope.

The present guide device also minimizes the stretching of the sigmoid colon and mesentery attached thereto even when the endoscope forms a loop in the sigmoid colon, which is known to be closely associated with occurrence of patient discomfort. The present guide device also allows an endoscopist to apply pushing force that is stronger than that normally exerted by an endoscopist in a typical colonoscopy procedure wherein the upper limit on the magnitude of force is generally determined by the level of discomfort tolerable by the patient or the susceptibility of the colon wall to injuries caused by parts of advancing endoscope. The present guide device helps reduce the tendency of the flexible endoscope to bend in long linear parts of the colon, which improves the transfer of pushing force from the proximal to distal ends of the endoscope. The present guide device facilitates multiple insertion and withdrawal of the endoscope in a single colonoscopy procedure. Other benefits and advantages of the guide device would be appreciated by those skilled on the art based on the description provided herein.

In one embodiment a guide device for an endoscope includes a tubular member that is configured to be inflated and collapsed; a plurality of straight portions; and a plurality of looped portions. The plurality of straight portions and the plurality of looped portions are arranged in an alternating manner and together comprise the tubular member. The guide device is configured to receive an endoscope and guide the endoscope along a path within an organ.

In another embodiment, a method for viewing an inside of an organ includes inserting an endoscope that is encircled with a tubular member of a guide device, the tubular member being configured to be inflated and deflated, the tubular member having a plurality of straight portions and a plurality of looped portions; inflating at least a portion of the tubular member after at least the portion of the tubular member is inserted into the organ with the endoscope; and viewing the inside of the organ using the endoscope. The tubular member is in a deflated state and stored in a housing while the endoscope and the housing are being inserted into the organ. The tubular member is inflated after at least a portion of the tubular member is released from the housing.

In yet another embodiment, a device for viewing inside of an organ includes an endoscope for inserting and viewing inside of an organ; and a guide component provided around the endoscope to facilitate insertion of the endoscope within the organ. The guide component is configured to be expanded and collapsed. The guide component includes a plurality of support sections to provide columnar strength, and a plurality of shaped sections to provide flexibility to the guide component. The plurality of support sections and the plurality of shaped sections are arranged in an alternating manner. The support sections are substantially straight and may be referred to as a straight portion. The shaped section includes a looped or curved section and may be referred to a looped section. The guide component is configured to be a single-use component that is to be discarded after a single use.

### BRIEF DESCRIPTION OF THE DRAWINGS

The following exemplary figures are provided to supplement the description below and more clearly describe the invention. In the figures, like elements are generally designated with the same reference numeral for illustrative convenience and should be used to limit the scope of the present invention.

FIG. 1A is a perspective view of a configuration of a portion of the tubular member comprising a guide device in inflated state wherein straight and looped sections are alternately arranged according to one embodiment of the present invention.

FIG. 1B is a perspective view of another configuration of a portion of the tubular member comprising a guide device in inflated state wherein looped sections are successively arranged according to one embodiment of the present invention.

FIG. 2A is a perspective view of another configuration of a portion of the tubular member comprising a guide device in inflated state wherein one or more looped sections in an exemplary configuration as shown in FIG. 1A are substituted for looped sections with larger external dimensions according to one embodiment of the present invention.

FIG. 2B is a perspective view of another configuration of a portion of the tubular member comprising a guide device in inflated state wherein one or more looped sections in an exemplary configuration as shown in FIG. 1B are substituted for looped sections with larger external dimensions according to one embodiment of the present invention.

FIGS. 3A to 3F are cross-sectional views of exemplary tubular members comprising a guide device in inflated state according to embodiments of the present invention.

FIG. 4 is a perspective view of an exemplary profile support structure incorporated into a portion of the tubular member of non-circular cross-section to help maintain the cross-sectional profile of the tubular member according to one embodiment of the present invention.

FIG. 5A is a perspective view of an exemplary configuration of the supply tube assembly according to one embodiment of the present invention.

FIG. 5B is a perspective view of another exemplary configuration of the supply tube assembly according to one embodiment of the present invention.

FIG. 6 is a perspective view of an exemplary embodiment of the housing for the tubular member mounted tightly encircling the shaft of an endoscope ready to be introduced into the colon according to one embodiment of the present invention.

FIG. 7 is a schematic representation showing an endoscope and a guide device partially deployed in the colon according to one embodiment of the present invention.

### DETAILED DESCRIPTION OF THE INVENTION

Embodiments of the present invention relate to a guide device capable of assisting the insertion and maneuver of an endoscope and reducing patient's discomfort and possibility of injury to the wall during endoscopic examination and treatment of the lower gastrointestinal tract or other bodily passages.

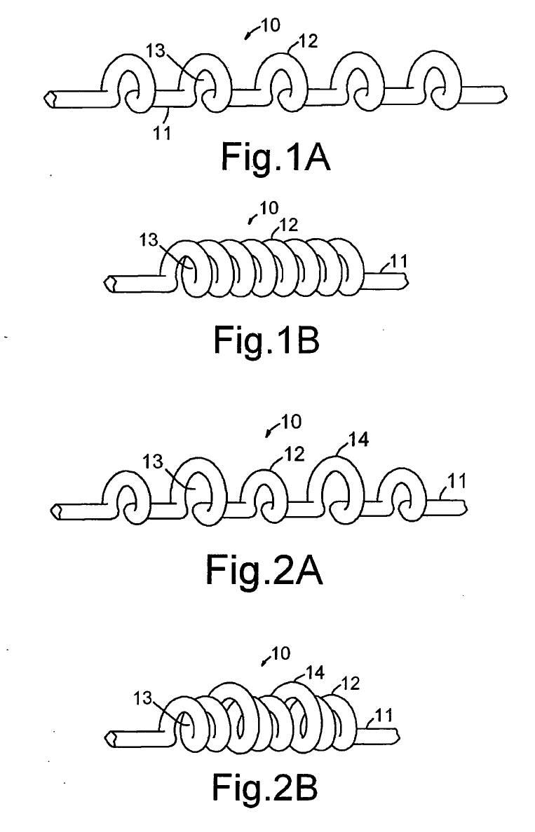

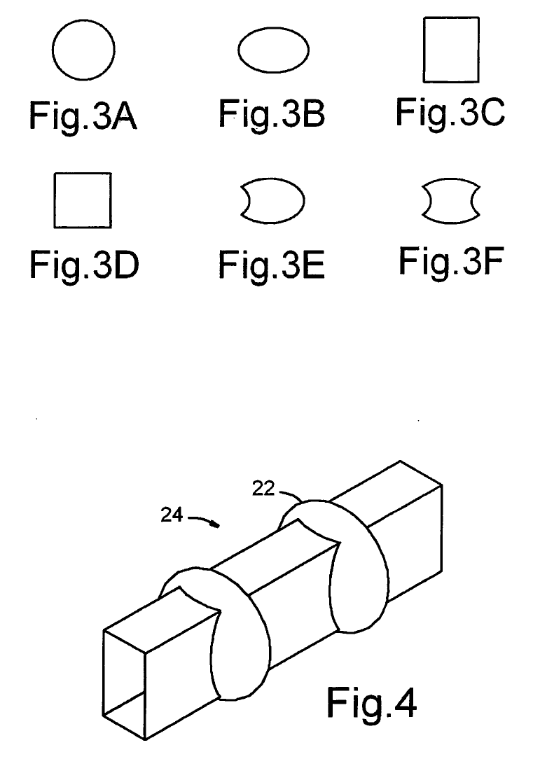

Referring to FIGS. 1A to 2B, exemplary configurations of a section of the tubular member 10 comprising the inflatable guide device according to embodiments of the present invention are shown in inflated state. The tubular member 10 generally comprises a continuous, collapsible tube, having a distal end and a proximal end, including straight 11 and looped sections 12 disposed in predetermined positions along the length thereof. An endoscope (not shown) is guided by the looped sections 12 through openings thereof 13, which keep moving shaft of endoscope from making contact with the colon wall throughout insertion process. The distal end of the tubular member is sealingly closed in the present embodiment.

The tube may be made of thin yet high tensile modulus, biocompatible polymer film material, preferably, with low frictional coefficient. Suitable construction materials may be, for example, polyethylene terephthalate (PET), polypropylene, polyamide (Nylon), and polyimide (Kapton). A suitable low friction coating (e.g., polytetrafluoroethylene (PTFE) or hydrophilic polymer) may be applied to the outer surface of the tube. Any suitable method may be employed to construct the shaped tubular member, for example, heat setting of a straight thermoplastic polymer tube into a desired shape or blow molding of a preconfigured thermoplastic polymer tube. In the present implementation, the wall thickness of the tube is no more than 0.1 mm and preferably, thinner than 0.05 mm. The thickness may vary according to application. Although these materials do not possess any structural qualities in film form, it is well known to those skilled in the art that pressure vessels of, for example, tubular or toroidal shapes constructed using thin film materials of high tensile modulus exhibit high enough structural strength for many practical applications when inflated even under moderate internal pressure. In use the internal pressure of the tubular member may be between 0.2 atmosphere and 8 atmosphere above ambient pressure. In one implementation, the internal pressure of the tubular member is between 0.3 atmosphere and 3 atmosphere above ambient pressure. In another embodiment, the internal pressure of the tubular member is more than 8 atmosphere above ambient pressure.

Still referring to FIGS. 1A to 2B, the straight sections 11 generally provide columnar strength whereas looped sections 12 provide flexibility to the tubular member 10. Due to the high tensile modulus of construction material and the configuration, the tubular member 10 in inflated state is highly resistive to stretching and compression. When inflated, the mechanical characteristics, such as flexural, compressional and torsional strengths, of a tube made of polymer film material, such as those listed previously, is closely correlated with the ratio of length to transverse dimension of the tubular member. By strategically arranging straight and looped sections of suitably chosen lengths and shapes, respectively, a tubular member section with fairly specific mechanical characteristics may be constructed.

FIGS. 1A and 2A show a configuration of a tubular member section wherein straight 11 and looped 12 sections are disposed mixedly. These configurations may be suitable for guiding an endoscope in linear parts of the colon, which help a flexible endoscope to remain straight under the bending load of two opposing forces between distal and proximal ends. Looped sections 12 in these configurations add some degree of flexibility to the otherwise rigid structure to lower the possibility of buckling, especially when the overall length of the tubular section becomes much larger than the transverse dimension thereof. Once a buckle develops in a section of a thin walled tubular member, it completely looses its mechanical strengths as a structural member.

Negotiating sharp bends in the colon require a tubular member configuration with omni-directional flexibility, which may be provided by a spiral configuration comprising several looped sections 12 joined successively without intervening straight sections, as shown in FIGS. 1B and 2B. Preferably, the size of the opening 13 of a looped section 12 is just large enough for an endoscope in use to pass freely while the external dimension is kept as small as practically possible without sacrificing the structural strength to minimize the amount of material needed for construction. Preferably, looped sections 14 with external dimensions large enough for outer side thereof to frictionally engage the colon wall are strategically disposed at predetermined positions along the length of the guide device to prevent the colon from sliding with respect to the guide device as shown in FIGS. 2A and 2B. A tubular sheath covering of an appropriate length with both ends fixedly attached to the tubular member or other suitable restraining means may be added to a section of the tubular member to limit the lengthwise stretching thereof.

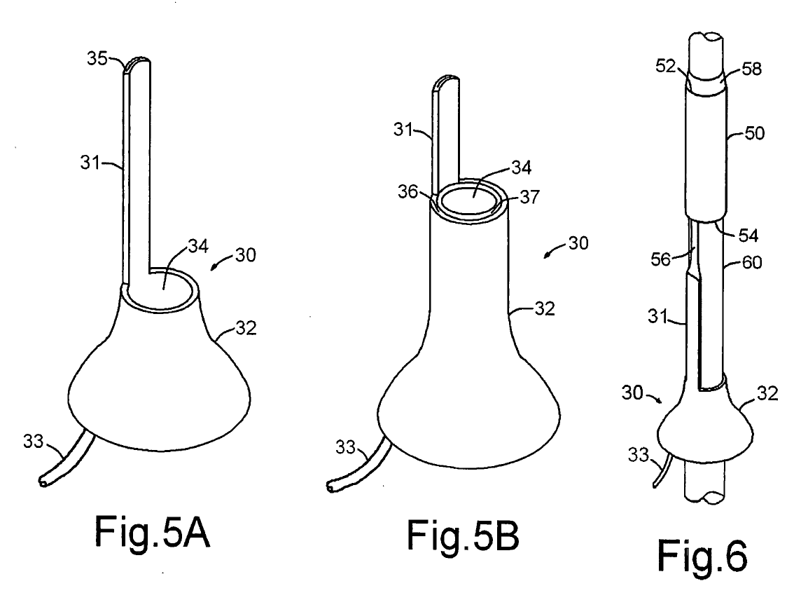

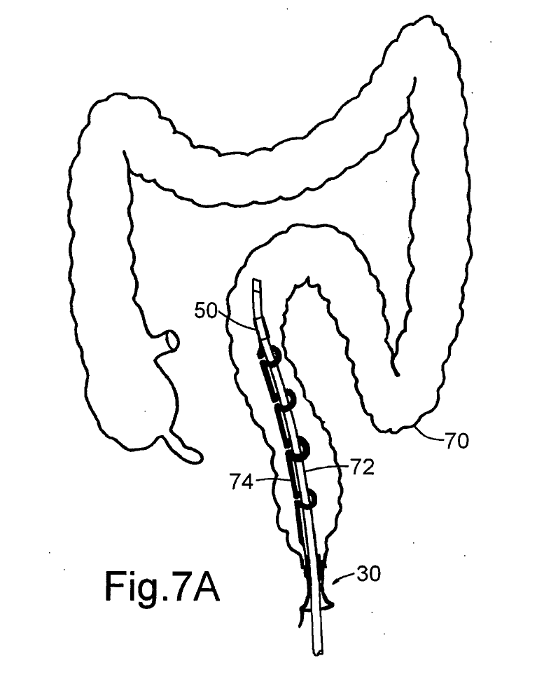

Another factor that influences the mechanical characteristics of the tubular member is the cross-sectional profile. Exemplary cross-sectional profiles of the tubular member in inflated state are shown in FIGS. 3A to 3F. The cross-sectional profile may be varied to enhance or fine-tune certain structural characteristics of a section of the tubular member, straight or looped. FIG. 4 shows an exemplary profile support structure 22 in inflated state that may be incorporated in several positions along a section of the tubular member 24 with non-circular cross-sectional profile to prevent the internal pressure from distorting the cross-sectional profile.

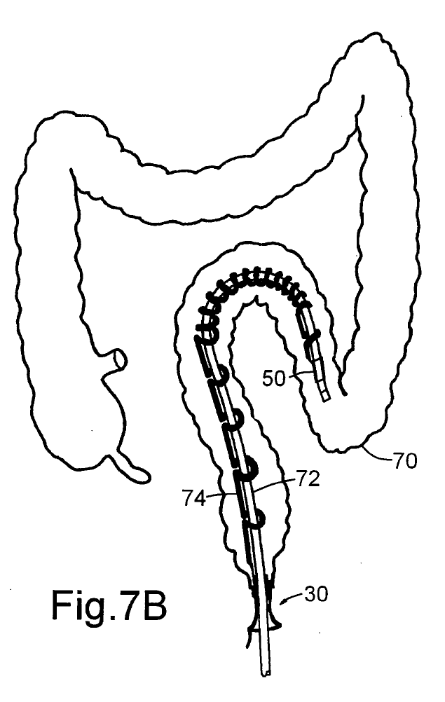

FIG. 5A is a perspective view of an exemplary configuration of a supply tube assembly 30. The supply tube assembly includes an internal 31 and an external 33 supply tubes and a funnel shaped body 32. There is provided a conduit in the funnel shaped body 32, which is in communication with the channels of internal 31 and external 33 tubes. A fluid may flow back and forth between the tubular member of the present guide device, the proximal end of which is attached fluid-tight to the internal supply tube 31 and the pressure controller unit (not shown) external to the colon, which is attached fluid-tight to the external supply tube 33. The internal supply tube 31 holds the proximal end of the tubular member immobile and causes the portion of the tubular member stowed collapsed in the housing to be sequentially released as the endoscope advances carrying the housing. Any suitable method, such as, adhesive bonding, welding, heat sealing or ultrasonic sealing may be used for joining the internal supply tube 31 and the tubular member.

Preferably, the internal supply tube 31 is of curved paddle shape to ease the introduction through the anus, piggybacking on the endoscope. Preferably, the curvature of the internal supply tube 31 is configured to allow it to be tightly fit around the shaft of the endoscope. The paddle shaped internal supply tube 31 may be thin, at most 1 mm thick, and long enough for the distal end 35 to completely pass through the anal canal and reach well into the rectum. Preferably, the internal supply tube 31 along with the rest of the assembly 30 is made of pliable polymer material with low friction coefficient to facilitate the movement of the endoscope as it pass through the anus sliding pressed against the internal supply tube 31. The cross-sectional area of the conduit in the internal supply tube may be varied by changing the overall width of the paddle along with the width of the conduit therein without increasing the thickness thereof. The contoured, funnel shaped body 32 remains outside of the anus rested against the patient body region surrounding the anus and acts as a fixed base for the internal supply tube 31 and the tubular member attached thereto and the external supply tube 33. The endoscope is introduced into the colon through the opening 34 in the body 32 of the assembly 30.

Alternatively, as shown in FIG. 5B, the length of the funnel shaped body 32 of the supply tube assembly 30 may be made long so that the distal end 36 thereof can be advanced into and disposed inside the rectum. In this configuration, the narrow part of the body 32 lines the anal canal completely holding the supply tube assembly 30 in position while the wide part remains outside the anus rested against the patient body region surrounding the anus. Preferably, the opening 34 of the body 32 is just large enough for the endoscope to pass through unrestricted. In this arrangement, the endoscope can enter or leave the colon passing through the anal canal without being constricted by the surrounding sphincter muscle. An annular seal 37 made of an elastic material, such as silicon rubber, may be disposed at the distal end 36 of the body 32 for sealingly and slidiably engaging endoscopes of various diameters.

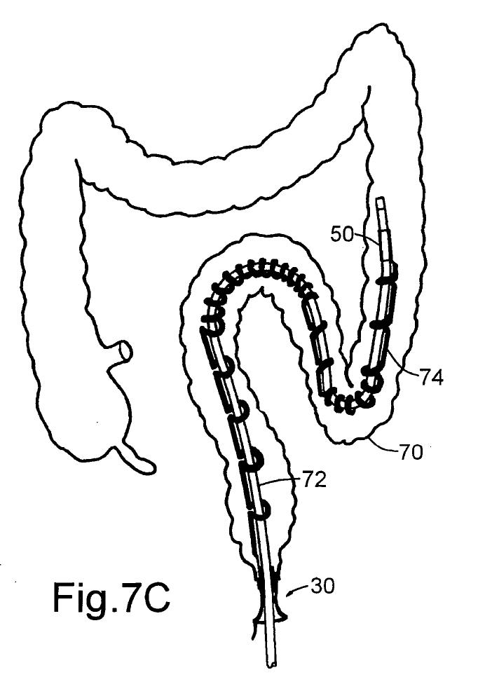

FIG. 6 shows a perspective view of an exemplary housing 50 for the present guide device mounted tightly encircling the shaft of an endoscope 60 and an exemplary embodiment of the supply tube assembly 30 with the internal 31 and the external 33 supply tubes and the funnel shaped body 32 prior to the insertion into the colon. The deflated and pleated portion of the tubular member comprising the present guide device is stowed sandwiched between a pair of thin polymer films comprising the housing 50 while the endoscope advances along the colon. A pair of thin polymer films with material properties very similar to the ones used to construct the tubular member comprising the present guide device may be used to construct the housing 50. The distal end of the housing 52 is closed and the proximal end 54 is left open wherefrom the pleated tubular member 56 slides out.

Preferably, the annular gap between the two films, wherein the pleated portion of tubular member is stored, is at most 1 mm wide and preferably, narrower than 0.5 mm. The length of the housing 50 may be long enough for the entire guide device to be stowed deflated, for example, around 10 cm. In use, the housing 50 is mounted tightly encircling the shaft of an endoscope 60 near the distal end using, for example, a locking collar 58, which holds the housing in place. Preferably, the locking collar 58 is made of a smooth polymer film material to minimize the cross-sectional profile to facilitate the passage of the endoscope and the housing through the anus.

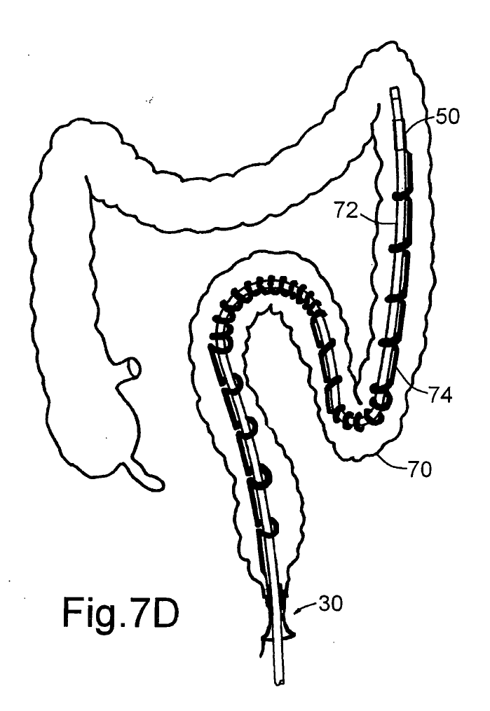

FIG. 7 schematically shows an endoscope 72 and the present guide device 74 partially inserted and deployed in the colon 70. Also shown in the figure are the housing 50, the supply tube assembly 30.

The procedure for using the present guide device is as follows. An endoscope with the guide device housing mounted tightly encircling the shaft near the distal end is inserted into the colon through the anus. As the endoscope advances along the colon, the tubular member comprising the present guide device in folded state sequentially slides out of the housing in step with the advancing endoscope. The freed portion of the tubular member is inflated into a preconfigured shape by the pressurizing fluid supplied through the conduit in the supply tube assembly. At a point where the depth of endoscope insertion exceeds the overall length of the tubular member, the tubular member becomes completely loose from the housing and assumes its full configuration.

During the withdrawal of the endoscope the pull string attached to the distal end of the tubular member is pulled in step with the withdrawing endoscope to sequentially deflate the tubular member starting from the distal end. Alternatively, the distal end of the tubular member may be predisposed to remain tethered close to the housing after leaving the housing so that it could be sequentially deflated in step with the withdrawing endoscope. During withdrawal the relief valve attached to the supply tube assembly is preset to maintain a predetermined constant pressure in the tubular member. When the withdrawal process reaches the rectum the endoscope is first pulled out of the colon and subsequently the fully deflated tubular member is withdrawn. The endoscope is reinserted into the rectum to examine the area of the rectum blocked from view by the crumpled tubular member. Alternatively, the rectum may be first examined soon after its introduction into the rectum prior to commencing the advancement of the endoscope. The present guide device may also be left in its fully inflated configuration for repeated insertion and withdrawal of the endoscope during procedures requiring multiple accesses to a site of interest within the colon.

While preferred illustrative embodiments of the invention are described above, it will be apparent to those skilled in the art that various changes and modifications may be made therein without departing from the invention. Accordingly, the appended claims should be used to interpret the scope of the present invention.

What is claimed is:

1. A guide device for an endoscope, the guide device comprising:
a tubular member that is configured to be inflated and collapsed;
a plurality of straight portions; and
a plurality of looped portions,
wherein the plurality of straight portions and the plurality of looped portions are arranged in an alternating manner and together comprise the tubular member, and
wherein the guide device is configured to receive an endoscope and guide the endoscope along a path within an organ.

2. The guide device of claim 1, wherein the tubular member is configured to be inflated once the tubular member is placed within the organ, wherein the guide device is configured to be a single-use component that is to be discarded after a single use.

3. The guide device of claim 2, wherein the tubular member is inflated using fluid.

4. The guide device of claim 3, wherein said fluid is air, nitrogen, or carbon dioxide.

5. The guide device of claim 3, wherein said fluid is water or saline solution.

6. The guide device of claim 3, wherein said fluid is pressurized at a pressure between 0.2 atmosphere and 8 atmosphere.

7. The guide device of claim 1, wherein said straight portion has a length in the range between 20 mm and 60 mm when fully inflated.

8. The guide device of claim 1, wherein the opening of said looped portion has a size in the range between 10 mm and 20 mm when fully inflated.

9. The guide device of claim 1, wherein said looped portion has an outer dimension in the range between 15 mm and 30 mm when fully inflated.

10. The guide device of claim 1, wherein said tubular member has the dimension of the cross sectional profile in the range between 5 mm and 20 mm when fully inflated.

11. The guide device of claim 1, wherein said tubular member has a wall thickness of no more than 0.05 mm.

12. The guide device of claim 1, wherein said tubular member is made of polyethylene terephthalate.

13. The guide device of claim 1, wherein said tubular member is made of polypropylene or polyamide.

14. The guide device of claim 1, wherein said tubular member is coated with polytetrafluoroethylene or hydrophilic polymer.

15. A method for viewing an inside of an organ, the method comprising:
inserting an endoscope that is encircled with a tubular member of a guide device, the tubular member being configured to be inflated and deflated, the tubular member having a plurality of straight portions and a plurality of looped portions;
inflating at least a portion of the tubular member after at least the portion of the tubular member is inserted into the organ with the endoscope; and
viewing the inside of the organ using the endoscope.

16. The method of claim 15, wherein the tubular member is in a deflated state and stored in a housing while the endoscope and the housing are being inserted into the organ, wherein the tubular member is inflated after at least a portion of the tubular member is released from the housing.

17. The method of claim 16, wherein the plurality of straight portions and the plurality of loop portions are arranged in an alternating manner, wherein the guide device is configured to be a single-use component that is to be discarded after a single use.

18. The method of claim 16, wherein the looped and straight portions of the tubular member in a folded state and are provided in a housing of the guide device.

19. The method of claim 18, wherein the looped and straight portions sequentially slide out of the housing as the endoscope advances into the organ.

20. The method of claim 19, wherein a freed portion of the tubular member that slid out of the housing is inflated using pressurizing fluid.

21. A device for viewing inside of an organ, the device including:
an endoscope to insert and view inside of an organ; and
a guide component provided around the endoscope to facilitate insertion of the endoscope within the organ, the guide component being configured to be expanded and collapsed, the guide component including
a plurality of support sections to provide columnar strength, and
a plurality of shaped sections to provide flexibility to the guide component.

22. The device of claim 21, wherein the plurality of support sections and the plurality of shaped sections are arranged in an alternating manner.

23. The viewing device of claim 22, wherein the guide component is configured to be expanded by increasing the internal pressure therein once the guide component is placed within the organ.

24. The viewing device of claim 22, wherein the support sections are substantially straight and the shaped section includes a looped section.

25. The viewing device of claim 22, wherein the guide component is configured to be a single-use component that is to be discarded after a single use.
